(19) United States
(12) Patent Application Publication
(10) Pub. No.: US 2018/0030326 A1
(43) Pub. Date: Feb. 1, 2018

(54) HIGH-HEAT DELIVERY DEVICE

(71) Applicant: DSM IP Assets B.V., Heerlen (NL)

(72) Inventors: Katarina TOMIC, Echt (NL); Harold Geert Hendrik VAN MELICK, Echt (NL); Travis BAUGHMAN, Echt (NL); Robin Elisabeth Maria Jacobus DAENEN, Echt (NL); Alexander Antonius Marie STROEKS, Echt (NL); Franciscus Johannes Marie DERKS, Echt (NL); Pieter GIJSMAN, Echt (NL)

(21) Appl. No.: 15/552,610

(22) PCT Filed: Feb. 26, 2016
(86) PCT No.: PCT/EP2016/054108

§ 371 Date: Aug. 22, 2017

(30) Foreign Application Priority Data
Feb. 26, 2015 (EP) 15156797.1

(51) Int. Cl. C09K 5/14; C08L 67/02; C08L 77/00; A61K 8/85; A61K 8/25; A61K 8/88; A61K 8/81; A61K 8/19; A61Q 5/06; A45D 2/00; A45D 19/16; A45D 1/00; A45D 7/02; D06F 75/00; C08K 3/04

(52) U.S. Cl. CPC C09K 5/14; C08K 3/04; C08L 67/02; C08L 77/00; A61K 8/85; A61K 8/25; A61K 8/88; A61K 8/8117; A61K 8/19; A61Q 5/06; A45D 2/001; A45D 19/16; A45D 1/00; A45D 7/02; D06F 75/00; C08L 2201/08; A61K 2800/87

(57) ABSTRACT

The present invention relates to a High-heat-delivery device suitable for use in an hair straightener, a hair curling iron or an ironing apparatus and suitable for heat transfer to a substrate by direct contact with said substrate, comprising at least one element (A) having a heat transfer contact surface area, wherein the element (A) comprises a thermo-conductive thermoplastic polymer composition comprising: 30 to 89 wt % of a polyester with a Tm of at least 170° C. as measured according to ISO 11357-1/3 (2011) with a scan rate of 10° C./min; and 10 to 50 wt % of glass fibers; and 1 to 40 wt % of a thermo-conductive additive, wherein the weight percentages are based on the total weight of thermo-conductive thermoplastic polymer composition of element (A).

The present invention relates to a high-heat delivery device. Furthermore, the present invention relates to an ironing apparatus, a hair straightener and a hair curling iron comprising the high-heat delivery device.

An efficient heat transfer to a substrate is difficult to carry out. Suitable heating may require very high temperatures if the heat transfer is not efficient enough. Most heating apparatuses comprise a heated metallic plate brought into contact with a substrate to be heated.

Transferring heat onto a substrate can damage the substrate. In particular, contacting a substrate at high temperatures, such as temperatures above 150° C. can cause burns to the substrate. A particular example is the application of hair straighteners, wherein hair to be straightened is submitted to high temperatures and result in substantial damaging of the hair, such as scorching, split ends, breakage, sharp bends, etc.

U.S. Pat. No. 6,622,735 describes a hair iron comprising a hair heating surface and a pressing surface of an iron covered with iron press covers made from a woven or nonwoven cloth of heat-resistant synthetic fibers.

US 2008/0041408 recites a hair styling apparatus that has a heating member and a polytetrafluoroethylene cloth covering the heating member.

It is thus an object of the present invention to provide a high-heat delivery device suitable for use in an hair straightener, a hair curling iron or an ironing apparatus and suitable for heat transfer to a substrate by direct contact with said substrate, which efficiently transfers heat at temperatures equal to or above 150° C. to a substrate while causing less damage to the substrate.

The object is met by a high-heat-delivery device, comprising at least one element (A) having a heat transfer contact surface area,

wherein the element (A) comprises a thermo-conductive thermoplastic polymer composition comprising:
30 to 89 wt % of a polyester with a Tm of at least 170 ° C. as measured according to ISO 11357-1/3 (2011) with a scan rate of 10° C./min; and
10 to 50 wt % of glass fibers; and
1 to 40 wt % of a thermo-conductive additive,
   wherein the weight percentages are based on the total weight of thermo-conductive thermoplastic polymer composition of element (A). Preferably the polyester has a Tm of at least 180° C., more preferably at least 200° C. and more preferred at least 210° C., even more preferred at least 230° C., and most preferred at least 240° C., as this allows higher application temperatures.

When a device is placed into or onto a hair styling apparatus, the device provides the advantage of reducing physical damages to the hair caused by styling at high temperatures, such as split ends, dryness, breakage. Further because the device according to the present invention is made of elements as described herein, the heat is more controllably transferred to the substrate.

In the context of the present invention, the term ‘high-heat’ is to be understood as a an application temperature which lies below the melting temperature of the polyester present in the thermo-conductive thermoplastic polymer composition of element (A). Preferably, the application temperature lies 20° C. below the melting temperature of the polyester. The application temperature may be for example at least 150° C., preferably 170° C., more preferably at least 180° C. and most preferred at least 190° C. An upper limit for the temperature for these applications usually is at most 240° C., preferably at most 230° C. and most preferred at most 220° C., and depends on the substrate to be treated.

In the context of the present invention, the high-heat delivery is to be understood as the delivery of heat through the device onto a substrate by direct contact with the substrate. The device according to the present invention is therefore part of a heating device.

In the context of the present invention, the term ‘device’ is to be understood as comprising the at least one element according to the present invention. In particular, the device may be designated as a sleeve having a substantially rectangular shape, a substantially cylindrical shape, a substantially triangular shape or any geometrical shape suitable to fix the device into or onto an apparatus for providing heat, such as a hair styling apparatus, or an iron for removing wrinkles from fabrics.

The device provides the advantage that the heat is transferred from the hair styling apparatus, or iron for removing wrinkles from fabrics, through the device and therefore transfers the heat to the substrate (such as hair or a piece of fabric). The advantage of the high-heat delivery device according to the present invention is that high-heat is transferred to the substrate while less damaging occurs, such as burning or any damage caused by excessive temperature, the substrate.

The term ‘by direct contact’ is to be understood in the context of the present invention as a contact without any intermediary layer between the device according to the present invention and the substrate to be heated. Accordingly, the device according to the present invention enters in contact with the substrate to which the heat has to be transferred. Accordingly, the device according to the present invention transfers the heat to the substrate via direct contact from the substrate, without an intermediary cloth between the device and the substrate.

In the context of the present invention, the substrate has a surface to be heated. The substrate can be of any material. When the high-heat delivery device is part of a hair styling apparatus, such as a hair straightener, a hair curling iron, or a hair waving apparatus, the substrate is hair. In the context of the present invention, the device being part of a hair styling apparatus allows the direct contact with the hair to style, dry, straighten, or wave without damaging the hair. The device can be applicable into or onto the hair styling apparatus. The hair may be clamped between two devices according to the present invention when the device is part of a straightening iron. The hair may be fixed around the device if the device is a cylinder piece of a curling iron.

When the high-heat delivery device is used in an ironing apparatus, the substrate is a fabric, or a piece of clothing from which wrinkles have to be removed.

In the context of the present invention, the device comprises at least one element, such as one element, two elements, three elements, four elements or more.

The device according to the present invention has the effect of efficiently providing the required heat to a substrate while causing less damage to said substrate.

In one embodiment of the present invention, the device further comprises at least one element (B) having a heat transfer contact surface area, the element (B) comprising a polymer composition comprising at least one polymer and at least one thermo-releasable substance. In this embodiment, an element (B) together with element (A) of the device, allows the release of a substance onto, or into, the substrate. The release of the thermo-releasable substance is carried out with the heating of element (B). The term ‘thermo-releasable’ is to be understood as the release of a substance under the effect of heat. Accordingly, the device according to this embodiment, provides heat to a substrate through elements (A) and (B), which are heated.

The elements have a surface area which enters in contact with the substrate and transfers the heat. In the present invention, the surface area is to be understood as the total area of the faces and curved surface of the element.

The total thickness of the device (thickness of elements (A) and/or (B)) is advantageously in the range 0.5 to 5 mm, more advantageously 0.7 to 3 mm, most advantageously 1 to 2 mm. This particular thickness range provides the advantage of having particularly efficient heat delivery.

In another embodiment according to the present invention, element (B) comprises a thermo-conductive additive. Element (B) accordingly comprises a thermo-conductive thermoplastic polymer composition.

In yet another embodiment according to the present invention, element (A) comprises at least one thermo-releasable substance.

When the composition is a thermo-conductive thermoplastic polymer composition comprising a thermo-releasable substance, the release of the thermo-releasable substance is carried out in a homogeneous manner onto, or into, the substrate. The presence of a thermo-conductive polymer with a thermo-releasable substance is advantageous when simultaneously release of the substance and heating of the substrate is desired. When the composition of at least one of elements of the device comprises a substance which is thermally releasable, the substance is simultaneously released when heat is transferred to the substrate and can further provide care to the substrate.

In the context of the present invention, the presence of a second element (B) allows the device to comprise two elements having different composition.

In one embodiment of the present invention, element (A) and/or element (B) further comprises at least one thermo-releasable substance. The released thermo-releasable substrate, such as fragrance, oil or another care substance provided to the substrate allows treating the substrate. Therefore, in this embodiment, the device allows heating of the substrate while damaging the substrate less, but it also provides care to the substrate by the heating of the device. In the context of the present invention, the substrate can therefore be treated by the thermo-releasable substance, while the heat is transferred. The treatment or care of the substrate may render an additional treatment of the substrate unnecessary. Therefore, when using a hair styling apparatus comprising the high-heat delivery device according to the present invention, the released substance conditions he hair, so that no additional protective hair spray is required before styling.

In another embodiment of the present invention, element (B) is partially embedded in element (A). The term ‘embedded’ is to be understood as being “physically included into”, which may be done by for example overmolding or 2K-molding. In the context of the present invention, the at least one element (B) may be at least partially included into element (A), thus at least a portion of (B) provides a heat transfer contact surface area with the substrate.

Element (A) comprises a thermo-conductive thermoplastic polymer composition comprising a polyester with a Tm of at least 170° C. as measured according to ISO 11357-1/3 (2011) with a scan rate of 10° C./min in an amount of between 30 wt % and 89 wt %; and glass fibers in an amount of between 10 wt % and 50 wt %; and a thermo-conductive additive in an amount of between 1 wt % and 40 wt %, wherein the weight percentages are based on the total weight of thermo-conductive thermoplastic polymer composition of element (A).

A polyester is hereby understood to include homopolyesters, thus having essentially the same building blocks, as well as copolyesters having different building blocks. The building blocks of copolyesters are also referred to as soft and hard blocks, in which the hard blocks may be polyester blocks and the soft blocks may be chosen from for example polyether, polycarbonate, dimer fatty acid dimer fatty diamine. Copolyesters have a Tm which depends on the type of soft block and molecular weight of the soft block and may for example vary between 170° C. and 220° C. A person skilled in the art may choose a suitable copolyester for application in the current invention based on the melting temperature of the copolyester.

Preferably the Tm of the polyester is at least 180° C., more preferably at least 190° C. even more preferred 200° C., most preferred at least 210° C. This allows higher application temperatures of the high-heat delivery device.

Examples of polyesters include poly butylene terephtalate (PBT), poly ethylene terephtalate (PET), poly ethylene naphtalate (PEN) and poly cyclohexanedimethylene terphtalate (PCT) and poly trimethylene terphtalate (PTT), as well as copolymers thereof, preferably the polyester is PET, as this allows for application temperatures in the range of 200° C. to 220° C., which is ideally for hair straighteners.

With the term melting temperature Tm is hereby understood the peak temperature of the endothermic melting peak measured in step [5] by DSC by the method according to ISO 11357-1/3 (2011) with a scan rate of 10° C./min, which includes the following steps:

A sample is first heated in a conventional differential scanning calorimeter (DSC) with a heating rate of 10° C./min in which a first melting temperature [Tm1] is defined, herein understood as the peak temperature of the highest endothermic melting peak. The instrument is purged with dry nitrogen gas with a purge flow 50 ml/min. In order to measure the melting temperature of the polyester, Tm, the following standard temperature program is applied:
[1] Heat from 20° C. to Tm1+20° C. at a scan rate of 10° C./min; purge gas N2 50 ml/min
[2] Keep at Tm1+20° C. for 3 min; purge gas N2 50 ml/min
[3] Cool from Tm1+20° C. to 20° C. at a scan rate of 10° C./min; purge gas N2 50 ml/min
[4] Keep at 20° C. for 5 min; purge gas N2 50 ml/min
[5] Heat from 20° C. to Tm1+20° C. at a scan rate of 10° C./min; purge gas N2 50 ml/min.
   The melting temperature Tm is defined as the peak temperature of the endothermic melting peak measured in step [5] by DSC by the method according to ISO 11357-1/3(2011) with a scan rate of 10° C./min.

Element (B) comprises a thermoplastic polymer composition comprising at least one thermoplastic polymer, at least one thermo-releasable substance and optionally at least one thermo-conductive additive. The thermoplastic polymer of element (B) may be selected from polyamides; polyesters; polyarylene sulfides such as polyphenylene sulfides; polyarylene oxides such as polyphenylene oxides; polysulfones; polyarylates; polyimides; poly(ether ketone)s such as polyetheretherketones; polyetherimides; polycarbonates, copolymers of said polymers among each other and/or with other polymers, including thermoplastic elastomers such as copolyetherester block copolymers, copolyesterester block copolymers, and copolyetheramide block copolymers; and mixtures of said polymers and copolymers. The thermoplastic polymer suitably is an a semi-crystalline or a liquid crystalline polymer, a thermoplastic elastomer, or a combination thereof.

Preferably, the thermoplastic polymer of element (B) also comprises the polyester as present in the thermo-conductive thermoplastic polymer composition of element (A), as this allows for better adhesion.

Accordingly, the device may comprise at least two elements, one of which may release a substance when providing heat to the device.

The thermoplastic polymer composition in elements (A) and/or (B) is preferably an halogen-free thermoplastic polymer, such as fluorine-free or chlorine-free thermoplastic polymer.

In the context of the present invention, the thermo-conductive thermoplastic polymer compositions comprise a thermo-conductive additive.

A thermo-conductive additive within the meaning of the present invention includes any material that can be dispersed in the at least one thermoplastic polymer and has a thermal conductivity λ (W/m·K) that is at least 5 times, preferably at least 10 times, more preferably at least 25 times higher than the thermal conductivity of the at least one thermoplastic polymer.

Thermo-conductive additives are known to a person skilled in the art and include metals, ceramics or carbon. Specifically, examples of common thermally conductive additives include aluminium, alumina, copper, magnesium, brass, carbon such as carbon nano tubes, carbon black and graphite, silicon nitride, aluminium nitride, boron nitride, zinc oxide, glass, mica, titanium oxide, calcium oxide and boron carbide. Preferably, the device according to the present invention comprises graphite, more preferably expanded graphite.

Preferably the thermo-conductive additive is in the form of a graphite powder having the shape of thin platelets. Platelets are herein understood to be particles having a flat shape with a large difference in the three dimensions such that the smallest dimension (the thickness) is much smaller than the size of the particles in the other two dimension (the length and the width). The platelets may consist of one or more individual graphite layers closely packed together.

The thickness (smallest dimension) of the platelets is less than 500 nm, preferably less than 200 nm, more preferably less than 100 nm, even more preferably less than 80 nm, and most preferably less than 50 nm. For the sake of clarity it is noted that the platelets do not need to have a flat structure. Due to the very low thickness these might be bended, curved or waved or deformed otherwise as well.

The thickness of the platelets can be determined by standard methods, such as by electron microscopy.

The term graphite powder comprising platelets of less than 500 nm, if not indicated otherwise, this is intended to include the preferred embodiments of thinner platelets as well. This graphite powder will also be indicated as graphite powder in the form of thin platelets.

The thermo-conductive additive being graphite powder surprisingly imparts to a thermoplastic polymer a relatively high thermal conductivity already at a relatively low weight percentage. It is sufficient to load the thermoplastic polymer with 1 to 40% by weight of the graphite powder, based on the total weight of the polymer composition, in order to obtain a polymer composition with reasonable thermal conductivity values. Preferably, the thermally conductive polymer composition according to the present invention comprises 3 to 30% by weight of the graphite powder, based on the total weight of the polymer composition, and more preferably 3 to 20 wt % by weight. Due to the relatively low amount of thermo-conductive additive the present polymer composition has better flow and mechanical properties than most known thermally conductive polymer compositions requiring high amounts of thermo-conductive additive in order to obtain reasonable conductivity values.

Typically, the graphite powder used in the thermo-conductive thermoplastic polymer composition is characterized by a relatively high specific surface area in combination with a relatively large particle size. Preferably, the graphite powder has a BET specific surface area of at least 10 m²/g and a particle size distribution characterized by a D(v, 0.9) of at least 50 μm. Normal graphite powders including powders of both synthetic graphites and natural graphites have either a high specific surface area in combination with a low particles size or vice versa a low specific surface area in combination with a large particles size.

The graphite powder used in the thermo-conductive thermoplastic polymer composition preferably has BET specific surface area of at least 10 m²/g, more preferably at least 15 m²/g, even more preferably at least 20 m²/g, and most preferably at least 25 m²/g. The BET specific surface area is determined according to ASTM D3037.

The present graphite powder preferably has a particle size distribution characterized by a D(v, 0.9) of at least 50 μm, more preferably at least 60 μm, even more preferably at least 70 μm, and most preferably at least 80 μm. In some embodiments, the particle size distribution is further characterized by a volume median diameter D(v, 0.5) of at least 20 μm, preferably at least 25 μm, more preferably at least 30 μm, and most preferably at least 35 μm. Moreover, the particle size distribution is usually characterized by a D(v, 0.1) of at least 6 μm, preferably at least 7 μm, more preferably at least 8 μm, and most preferably at least 9 μm. In one embodiment the graphite powder has a particle size distribution characterized by a D(v, 0.9) of at least 50 μm, a volume median diameter D(v, 0.5) of at least 20 μm, and a D(v, 0.1) of at least 6 μm. The particle size is not bound by a specific maximum limit, but in practice it will be limited by the minimum specific surface area required. The higher the minimum specific surface area the smaller the particles in general will be and also the smaller the maximum size of the particles.

D(v, 0.9), D(v, 0.5), and D(v, 0.1) are determined by laser diffraction using a Malvern Mastersizer.

Typically, the graphite powder has a xylene density in the range of 2.0-2.4, preferably 2.1-2.3 g/cm³, more preferably 2.20-2.27 g/cm³. A graphite powder suitable as thermo-conductive additive in the thermally conductive polymer composition is available from TIMCAL Ltd., Bodio, Switzerland under the trade name TIMREX® BNB90.

The thermo-conductive thermoplastic polymer compositions may optionally comprise any auxiliary additives, known to a person skilled in the art that are customarily used in polymer compositions. Preferably, these other additives should not detract, or not in a significant extent, from the invention.

Such additives include non-conductive fillers; pigments; dispersing aids; processing aids for example lubricants, mould release agents, flow additives; impact modifiers; plasticizers; crystallization accelerating agents; nucleating agents; flame retardants; UV stabilizers; antioxidants; heat stabilizers.

Such additives include, in particular, other fillers not considered thermally conductive such as non-conductive reinforcing fillers. The non-conductive fillers that may be used as additives to the thermally conductive polymer composition include non-conductive inorganic fillers. Suitable for use as non-conductive inorganic fillers are all the fillers, such as reinforcing and extending fillers, known to a person skilled in the art, for example asbestos, mica, clay, calcinated clay, talcum, silicates such as wollastonite, and silicon dioxide, especially glass fibres. In this context, “non-thermally conductive” or “non-conductive” is used to describe filler materials having an intrinsic thermal conductivity λ (W/m·K) that is less than 5 times higher compared to the thermal conductivity of the at least one thermoplastic polymer.

The device according to the invention comprises an element (A) which comprises a thermo-conductive thermoplastic polymer composition comprising glass fibers in an amount of 10 to 50 wt %, with respect to the total weight of composition, preferably 15 to 40 wt %, and more preferably 20 to 40 wt %. The presence of glass fibers ensure sufficient mechanical properties.

In the context of the present invention, the at least one elements of the device comprises a thermo-conductive thermoplastic polymer composition having a through-plane thermal conductivity of between 0.3 to 5 W/m·K, preferably between 0.7 to 3 W/m·K, and more preferably between 0.8 to 2.5 W/m·K.

In the context of the present invention, the device comprises a thermo-conductive thermoplastic polymer composition having a high thermal conductivity and good mechanical properties, which can vary over a wide range depending on the amount of the thermo-conductive additives, and optional other addtives. Due to the graphite powder in the form of thin platelets, already at very low total amount of thermally conductive additive, reasonably high thermal conductivities are obtained, which with many other thermally conductive fillers are reached at much higher contents and at the cost of mechanical properties. At higher total amounts of thermally conductive additives much higher thermal conductivities are obtained compared to other thermally conductive additives when used in similar amounts.

According to one embodiment of the present invention, the high-heat-delivery device element (A) constitutes 5-100% of the total heat transfer contact surface area of elements (A) and/or (B) and element (B) constitutes 95-0% of the total heat transfer contact surface area of elements (A) and/or (B). Advantageously, the high-heat-delivery device element (A) constitutes 20 to 80%, preferably 40-60%, most preferably 50% of the total heat transfer contact surface area of elements (A) and/or (B). Element (B) constitutes 20 to 80%, preferably 40-60%, most preferably 50% of the total heat transfer contact surface area of elements (A) and/or (B).

In one embodiment of the present invention, the through-plane conductivity of element (A) is in the range 0.6 to 3.5 W/mK (Watts per meter Kelvin), at a temperature of 200° C.,preferably 0.7 to 2.5 W/mK, more preferably 0.75 to 1.5 W/mK.

In one embodiment of the present invention, the through-plane conductivity of element (B) is in the range 0.3 to 2.5 W/mK at 200° C., preferably 0.6 to 2.5 W/mK.

In the context of the present invention, the term ‘at least one’ is to be understood as one, two, three, four, five, six or more.

In one embodiment of the present invention, element (B) is present in two separate portions. Accordingly, in this embodiment, element (B) comprises one element (A) and two elements (B) separate from each other.
Optionally, elements (A) and/or (B) comprise
(iii) 1-25 wt. % of a substance to be released, advantageously 1-20 wt. %, more advantageously 1-7 wt. % of the total weight of the composition.

Accordingly, in one embodiment of the present invention, the high-heat delivery device comprises at least two elements (element (A) and element (B)) made of different compositions. The device is suitable for heat transfer to a substrate by direct contact with said substrate,
wherein the high-heat-delivery device element (A) comprises a first thermo-conductive thermoplastic polymer composition comprising:

(a) at least a polyester with a Tm of at least 170° C. as described above ;

(b) at least one thermo-conductive additive;

(c) glass fibers;

(d) optionally a thermo-releasable substance;
wherein high-heat-delivery device element (B) comprises a second thermo-conductive thermoplastic polymer composition comprising:

(i) at least a polyester with a Tm of at least 170° C. as described above;

(ii) at least one thermo-releasable substance;

(iii) optionally at least one thermo-conductive additive.

The high-heat-delivery device according to the present invention is further characterized by being composed of elements made of a material having a reasonable mechanical performance, such as:
a tensile strength of at least 40 MPa, preferably at least 50 MPa and more preferably at least 60 MPa;
an elongation at break of at least 0.7%, preferably at least 1.0%, more preferably at least 1.5%, and most preferably at least 2.0%;
stiffness of at least 4,000 MPa, more preferably at least 6,000 MPa;
tensile modulus, tensile strength and elongation at break are determined at 23° C. and 5 mm/min according to ISO 527; the dried granulate of the thermoplastic material of the different elements of the device was tested after injection moulding to form the test bars for the tensile tests having a thickness of 4 mm conforming to ISO 527 type 1A.

In the context of the present invention, a thermo-releasable substance is a substance that can be released when heat is provided and includes cosmetically acceptable substances as known in the art, including mixtures. When the thermo-conductive thermoplastic polymer composition comprising a thermo-releasable substance is heated, said substance is released. In the high-heat delivery device, when the device is heated and transfers the heat onto the substrate by direct contact with said substrate, the substance is released onto the substrate. In the embodiments of the present invention, the thermo-releasable substance can be a fragrance and/or an oil. The fragrance provides a perfume or smell that is provided by release of the fragrance in the form of a vapour. The oil is released as a liquid onto the substrate and provides care or a treatment to the substrate.

In one embodiment of the present invention, the thermo-releasable substance can be selected from the group consisting of natural oils, such as argan oil, avocado oil, sunflower oil, jojoba oil, camealia oil; triglycerides; polydimethylsiloxane (PDMS); silicone oils; petroleum oils distillates, such as squalene, squalane, paraffin.

The advantage of the thermo-releasable substance which is an optional component of at least one of the elements of the device according to the present invention, is that no additional application of the substance is required onto the substrate or the device. Preferably, the thermo-releasable substance can be a treating substance, a personal care substance, and/or a substance providing vitamins, minerals, moisture, no prior application of any substance on the substrate or the device is required prior, during or after the use of the device.

According to the present invention, the device is made by injection molding. Accordingly, elements (A) and (B) are preferably injection moulded parts. Elements (A) and (B) can also be fabricated by 2K moulding, which is a known process per se, as well as overmoulding.

One aspect of the present invention relates to apparatuses comprising the high-heat-delivery device according to the present invention, such as hair styling apparatuses or ironing apparatuses.

The thermo-conductivity of the materials is measured as follow. The thermo-conductivity measurements are performed using a LFA447 Nanoflash from Netzsch. Samples are acquired from a 120×120×1mm injection moulded part and are cut from the middle of the moulded part, to avoid possible edge effects, which in turn might influence the thermal conductivity. With this type of geometry the orientation dependent thermal conductivity can generally be described with three parameters: Λ_{⊥}, Λ_{//} and Λ₊, wherein Λ_{⊥} is the through-plane thermal conductivity, Λ_{//} is the in-plane thermal conductivity in the direction of maximum in-plane thermal conductivity, and Λ_{±} is the in-plane thermal conductivity in the direction of minimum in-plane thermal conductivity. For the through plane measurements a standard geometry of 10 mm by 10 mm is used with a thickness of 1 mm.

The measuring method uses the so called flash method, in which a material is heated on one side by a flash lamp and its temperature profile is measured on the other side of the material with an Infrared Detector. The material is sprayed black in advance, by use of a graphite spray, in order to acquire a black radiator. From the fit parameters of the measured temperature profile the thermal diffusivity a(T) is calculated, several theoretical models are available. Mostly the so called Cowan-model will be used. For determining the Heat capacity of a sample it is necessary to measure a reference sample (with known thermal properties) in each measuring-cycle as well. Usually Pyroceram 9606 is used. The heat capacity can be calculated by comparing the peak-hight of the measured temperature profile of the unknown sample to that of the reference sample. The sample geometry should be as close as possible to the dimensions of the reference sample to get proper results for heat capacity (a reference square sample of 10 mm by 10 mm and with a thickness of 1 mm is used). The density of a sample is determined by measuring length, width and thickness as well as measuring the mass of the sample. From the thermal diffusivity (D), the density (ρ) and the heat capacity (Cp), the thermal conductivity of the material can be determined. For the through plane conductivity (λ_{⊥}) the following formula applies:

λ⊥=a⊥(T)*p*Cp
All measurements can be performed in the temperature range from ambient to 300° C.

The invention is illustrated by the following figures. The invention is further illustrated by examples and comparative experiments.

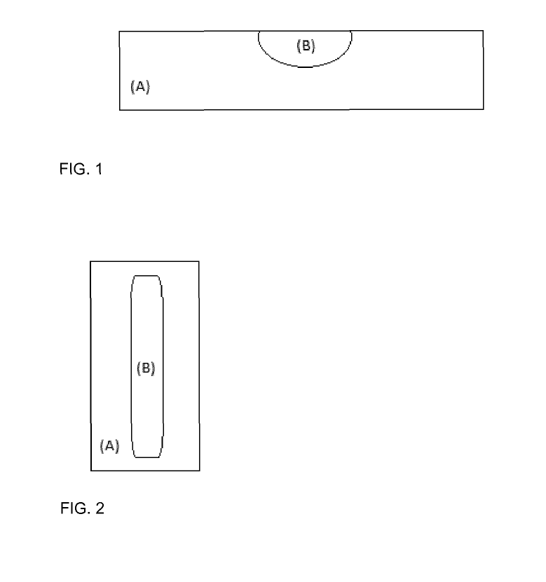

FIG. 1: high-heat delivery device (transverse view) according to the present invention comprising an element (A) and an element (B) as defined herewith

FIG. 2: high-heat delivery device (top view) according to the present invention comprising an element (A) and an element (B) as defined herewith

### EXAMPLES

### Materials Used:

Polymers:
PA-6=polyamide-6=Akulon K125 or Akulon K122 of DSM
PA-66=polyamide-6,6=S222 of DSM
PA-46=polyamide-4,6=KS200 of DSM
PA-4,10=polyamide-4,10=EcoPaxx Q150MS of DSM
SPS=Syndiotactic Polystyrene (Idemitsu Chemicals Europe, Xarec 90ZC, Xarec 300ZC, Xarec 142ZE)
PET (5018)=polyethyleneterephthalate=Amite® BAGA 5018 of DSM, Tm=254 ° C.
PBT-Eco=polybutyleneterephthalate-co-dimerfattyacid 20% DFA of DSM (Melt volume rate=3), Tm=210° C.
PBT-Eco-2=polybutyleneterephthalate-co-dimerfattyacid 20% DFA of DSM (Melt volume rate=25), Tm=209° C.
PBT-Eco-3=polybutyleneterephthalate-co-dimerfattyacid 40% DFA of DSM (Melt volume rate=40), Tm=180 ° C.
PBT-E=polybutyleneterephthalate-co-polytetramethyleneoxide=Arnitel® EL740 (Melt volume rate=15), Tm=221° C.
PBT=polybutyleneterephthalate, Tm=225° C.
PBN-D=polybutylenenaphthanate-co-dimerfattyacid amide=experimental product of DSM
Comp=Compatibilizer for PA with SPS: acid modified poly(phenylene ether) CX-1, FA-PPE (Idemitsu Kosan Co., Ltd)

### Thermo-Conductive Additives

Expanded Graphite (C-Therm 01)
Expanded Graphite (Ecophit GFG1200)
Carbon black (Timcal Ensaco260G)
Boron Nitride

### Oils:

Argan Oil=product code 50 3808 1 of DSM Nutritional Products
Avocado oil=Persea Gratissima oil, Avoaodo oil RBD, code 266554 of IMCD
Sunflower oil=product code 1665N of Volatile
Camealia Oleifera Seed Oil=Cropure Yuchayu-LQ-(JP), product code SV70391 of
Croda Jojoba oil=Simmondsia Chenensis Seed Oil=Cropure Jojoba of Croda

### Other Additives:

Irganox-1076=stabilizer Irganox1076 of Ciba (BASF)
Irganox-1098=stabilizer Irganox 1098 of Ciba (BASF)
Irganox-B1171=stabilizer Irganox B1171 of Ciba (BASF)
Glass=3B CS173-x11 of Owens Corning
HP 3786=Glass fibers of PPG Fiber Glass, average fiber diameter 10 micrometer.
Licolub WE40=ester of montanic acids; mold release agent (Clariant)
Table 1 shows various compositions according to the invention for which the thermo-conductivity was measured.

### Preparing Compositions

Various compositions were made according to the tables below. The compositions were compounded on twin screw extruders like ZSK30/44D at a processing temperature being at least equal to the highest of Tg or Tm of the polymer of the composition. After the mixing the hot polymer composition string was cooled in a water bath or cooling belt and cut into granules suitable for injection molding.

### Injection Molding

Injection molding was performed at an Engel 110, a machine with 110 ton maximum clamping force with a screw diameter of 30 mm. On this machine 120×120×1 mm plates were produced of almost all compositions using plate 120×120×1 mm, for some materials also mechanical test bars were produced on the Engel 110, for these test bars plate ISO 527-1A pr.80*10*4 2v. was used. From the plates plaques were cut to determine the release of the thermo-releasable substance from the composition when used on the straightener. Test bars (dog-bone) were used to produce stress-strain curves of various compositions. Tensile modulus was determined at 23° C. and 200° C. and 5 mm/min according to ISO 527; the dried granulate of the thermoplastic material of the different elements of the device was tested after injection moulding to form the test bars for the tensile tests having a thickness of 4 mm conforming to ISO 527 type 1A.

### Hair Temperature Measurements

A high-heat-delivery device being a hair straightener, comprising at least one element (A) according to the invention was tested on human hair to measure the heat transfer to the hair as compared to a comparative straightener with aluminum plates.

The application temperature for both straightener was 200° C. The measured temperatures on the hair immediately after applying the straightener and after a stroke of about 20 cm on the hair was measured for severl strokes and is given in table 3.

The straightener, listed in table 3, was heated to 200° C. Human hair swatches were used as test material being 1.5 cm wide, 23 cm free length held together with by a glued part which was not pulled through the straightener (Klebetresse dicht aus Euro-Natur-Haar, remis, Farbe 6/0, Kerling International Haarfabrik GmbH, item number 826500). A stroke is defined as pulling the hair swatch through the straightener in approximately 10 seconds per pull. A test method has been performed to measure hair temperature after straightening of the hair with straightener with aluminum plates (without sleeves; comparative example in Table 3) in comparison to the straightener with 2 plastic sleeves mounted on aluminum plates. The sleeves, each having a thickness of 1.5 mm were prepared by overmolding two compositions: element A having the composition as described in Example 3 of Table 1 and Element B having the composition A of Table 2. Hair was treated with a number of strokes as given in Table 3. The temperature of the hair was recorded at the beginning of a stroke and at the end in order to measure heat transfer.

TABLE 1
Compositions for element (A)

| | | | | | | | | | Tensile | |
|---|---|---|---|---|---|---|---|---|---|---|
| | | | Thermo- | | | | | Thermal | modulus | Tensile |
| | | | conductive | | Glass | Additive | | conductivity | at room | modulus at |
| Experi- | polymer | | additive | | fiber | mix | | at 200° C. | temperature | 200° C. |
| ment no | [wt %] | | [wt %] 1 | | [wt %] | [wt %] | | [W/(m*K)] | [MPa] | [MPa] |
| 1 | PET(5018) | 57.1 | C-therm 01 | 7.5 | 35 | 2 | 0.4 | 1.07 | 15431 | 4593 |
| 2 | PET(5018) | 59.7 | C-therm 01 | 9.9 | 30 | 2 | 0.4 | 1.22 | 14905 | 4716 |
| 3 | PET(5018) | 59.7 | C-therm 01 | 4.9 | 35 | 2 | 0.4 | 0.83 | 14911 | 4676 |
| 4 | PET(5018) | 65.8 | C-therm 01 | 7.5 | 25 | 2 | 0.4 | Similar to exp 1* | 12704 | 4098 |
| 5 | PET(5018) | 71.4 | C-therm 01 | 9.9 | 33 | 2 | 0.4 | Similar to exp 2* | 10747 | 3167 |

1 thermo-conductive additive is added as masterbatch PET (5018) 70 wt % and thermo- conductive additive 30 wt %, with respect to the masterbatch;
2 0.3 wt % LicolubWE40, 0.1 wt % Sodium benzoate
*Not measured, conductivity estimated based on the amount of thermo- conductive additive

TABLE 2
Compositions suitable for element (B)

| | | | | | | | | | | | | | | Conduc- | Conduc- |
|---|---|---|---|---|---|---|---|---|---|---|---|---|---|---|---|
| | | | | | | | Thermo- | | | | | | | tivity | tivity |
| | polymer | | polymer | | | | conductive | | | Other | | additive | | @ 25° | @ 200° |
| Experi- | type | | type | | oil type | | additive | | Glass | Additive | | Irganox | | C. [W/ | C. [W/ |
| ment no | [wt %] | | [wt %] | | [wt %] | | [wt %] | | [wt %] | [wt %] | | [wt %] | | (m*K] | (m*K] |
| A | PET | 43.24 | PBT- | 30.63 | mix 1 | 5.41 | C-Therm- | 20.00 | | | | 1076 | 0.72 | 0.97 | 1.03 |
| | BAGA | | Eco | | | | 001 | | | | | | | | |
| B | PET | 40.84 | PBT- | 34.71 | Avocado | 6.13 | C-Therm- | 17.50 | | | | 1076 | 0.82 | 0.67 | 0.68 |
| | BAGA | | Eco | | | | 001 | | | | | | | | |
| | (MB) | | | | | | | | | | | | | | |
| C | PET | 54.70 | PBT- | 22.10 | Avocado | 7.36 | C-Therm- | 15.00 | | | | 1076 | 0.84 | 0.51 | 0.50 |
| | BAGA | | Eco3 | | | | 001 | | | | | | | | |
| | (MB) | | PBT- | | | | | | | | | | | | |
| | | | Eco2 | | | | | | | | | | | | |
| | | | 50/50 | | | | | | | | | | | | |
| D | PET | 50.50 | PBT- | 27.77 | Avocado | 5.89 | C-Therm- | 15.00 | | | | 1076 | 0.84 | 0.75 | 0.76 |
| | BAGA | | Eco | | | | 001 | | | | | | | | |
| | (MB) | | | | | | | | | | | | | | |
| E | PA- | 38.94 | SPS | 23.80 | Argan | 9.38 | Ecophit | 14.25 | 10 | Comp | 2.16 | B1171 | 0.72 | 0.63 | 0.59 |
| | 4,10 | | Xarec | | | | GFG1200 | | | | | | | | |
| | | | 142ZE | | | | Timcal | 0.75 | | | | | | | |
| | | | | | | | ensaco260 | | | | | | | | |
| F | PA- | 36.35 | SPS | 22.21 | Argan | 8.75 | Boron | 20.00 | 10 | Comp | 2.02 | B1171 | 0.67 | 0.45 | 0.40 |
| | 4,10 | | Xarec | | | | Nitride | | | | | | | | |
| | | | 142ZE | | | | | | | | | | | | |
| G | PA- | 38.94 | SPS | 23.80 | Argan | 9.38 | Ecophit | 15.00 | 10 | Comp | 2.16 | B1171 | 0.72 | 0.54 | 0.50 |
| | 4,10 | | Xarec | | | | GFG1200 | | | | | | | | |
| | | | 142ZE | | | | | | | | | | | | |
| H | PBT | 53.47 | PBT- | 30.30 | Argan | 5.35 | Ecophit | 10.00 | | | | B1171 | 0.88 | 0.50 | 0.62 |
| | | | Eco2 | | | | GFG1200 | | | | | | | | |

mix 1 = 94/2, 5/2, 5/1 Avocado/Argan/Camelia/Vitamine-E

TABLE 3
Experiments on hair to measure heat transfer

| | | Example according to the invention |
|---|---|---|
| | | Straightener according to the |
| | Comparative | invention comprising two sleeves |
| | example | made from element A: example 3 |
| | Straightener with | from the table 1 and element |
| | aluminum plates | B: example A from Table 2 |
| Application | 200° C. | 200° C. |
| temperature of the | | |
| straightener | | |
| Measured hair | 155° C. | 143° C. |
| temperature 1^{st} | | |
| stroke beginning | | |
| Measured hair | 122° C. | 109° C. |
| temperature 1^{st} | | |
| stroke at the end | | |
| Measured | 150° C. | 140° C. |
| temperature 2^{nd} | | |
| stroke beginning | | |
| Measured hair | 132° C. | 120° C. |
| temperature 2nd | | |
| stroke at the end | | |
| Measured hair | 155° C. | 145° C. |
| temperature 3^{rd} | | |
| stroke beginning | | |
| Measured hair | 137° C. | 125° C. |
| temperature 3^{rd} | | |
| stroke at the end | | |

Table 1 shows compositions suitable for element (A) and Table 2 show compositions suitable for element (B) of the high-heat delivery device according to the invention; Comparative examples C_1 to C_3 exhibit less thermo-conductivity due to the absence of thermo-conductive additive.

The results of Table 3 clearly indicate that the hair straightener according to the invention can effectively transfer heat to hair, in a way almost similar to aluminium which is known to be of much better conductance as compared to a polyester composition according to the invention. Surprisingly, the hair straightener according to the invention showed very good straightening properties. A measure of straightening of hair is reflected by hair temperature after use of a straightener device and generally may be at least 120° C. to create proper straightening performance. Results are compared for the straightener without plastic sleeves and with sleeves comprising element A and B. Surprisingly, the straightener according to the invention was able to transfer heat in a way almost similar to a hair straightener with aluminum contact surface.

1. High-heat-delivery device suitable for use in an hair straightener, a hair curling iron or an ironing apparatus and suitable for heat transfer to a substrate by direct contact with said substrate, comprising at least one element (A) having a heat transfer contact surface area,
wherein the element (A) comprises a thermo-conductive thermoplastic polymer composition comprising:
30 to 89 wt % of a polyester with a Tm of at least 170° C. as measured according to ISO 11357-1/3 (2011) with a scan rate of 10° C./min; and
10 to 50 wt % of glass fibers; and
1 to 40 wt % of a thermo-conductive additive,
wherein the weight percentages are based on the total weight of thermo-conductive thermoplastic polymer composition of element (A).

2. Device according to claim 1, wherein the device further comprises at least one element (B) having a heat transfer contact surface, wherein element (B) comprises a thermoplastic polymer composition comprising a polymer and at least one thermo-releasable substance.

3. Device according to claim 2, wherein the thermoplastic polymer composition of element (B) comprises a thermo-conductive additive.

4. Device according to claim 1, wherein element (A) comprises at least one thermo-releasable substance.

5. Device according to claim 2, wherein element (B) is partially embedded in element (A).

6. Device according to claim 2, wherein the thermoplastic polymer composition of element (B) comprises a thermoplastic polymer selected from the group consisting of polyamides, polyesters, polyphenylene sulphides, polyphenylene oxides, polysulfones, polyarylates, polyimides, polyetheretherketones and polyetherimides, and mixtures thereof and copolymers thereof.

7. Device according to claim 2, wherein the heat transfer contact surface area of element (A) constitutes 20-80% of the combined heat transfer contact surface areas of (A) and (B) and element (B) constitutes 80-20% of the combined heat transfer contact surface areas of (A) and (B).

8. Device according to claim 1, wherein the thickness of element (A) and optionally element (B) is is 0.5 mm to 2.0 mm.

9. Device according to claim 1, wherein the through-plane conductivity of element (A) and/or the through-plane conductivity of element (B) is in the range 0.3 to 2.5 W/m K at 200° C.

10. Device according to claim 1, wherein at least one of element (A) and/or (B) comprises a thermo-conductive additive selected from the group consisting of graphite, expanded graphite, carbon black and boron nitride.

11. Device according to claim 1, wherein the thermo-conductive thermoplastic polymer composition of element (A) comprises
(i) 40 to 80 wt. % of polyesterterephtalate; and
(ii) 15 to 40 wt % glass fibers; and
(ii) 3 to 20 wt. % of a thermo-conductive additive,
wherein the weight percentage is with respect to the total weight of thermo-conductive thermoplastic polymer composition of element (A).

12. Device according to claim 2, wherein the thermoplastic polymer composition of element (B) comprises
(i) 30-85 wt. % of a polymer; and
(ii) 5-50 wt. % of a thermo-conductive additive and
(iii) 1-25 wt. % of a thermo-releasable substance
wherein the weight percentage is with respect to the total weight of thermoplastic polymer composition of element (B).

13. Device according to claim 2, wherein the thermo-releasable substance comprises a fragrance and/or an oil selected from the group consisting of argan oil, avocado oil, sunflower oil, jojoba oil and camealia oil, and mixtures thereof.

14. Device according to claim 2, wherein the thermoplastic polymer composition in of (B) comprises a polyester.

15. Device according to claim 1, wherein elements (A) and/or (B) are injection moulded parts.

16. Method for releasing thermo-releasable substance from a composition as defined in claim 2, comprising heating the thermo-conductive composition and thereby releasing said thermo-releasable substance.

17. Hair styling apparatus comprising the high heat delivery device according to claim 1.

18. Ironing apparatus comprising the high heat delivery device according to claim 1.
